(19) United States
(12) Patent Application Publication
(10) Pub. No.: US 2018/0155690 A1
(43) Pub. Date: Jun. 7, 2018

(54) METHOD FOR PREPARING NATURAL KILLER CELLS USING IRRADIATED PBMCS, AND ANTI-CANCER CELL THERAPEUTIC AGENT COMPRISING THE NK CELLS

(71) Applicant: KOREA INSTITUTE OF RADIOLOGICAL & MEDICAL SCIENCES, Seoul (KR)

(72) Inventors: You-Soo Park, Seoul (KR); Cheol-Hun Son, Seoul (KR); Kwangmo Yang, Seoul (KR)

(21) Appl. No.: 15/822,843

(22) Filed: Nov. 27, 2017

(30) Foreign Application Priority Data
Sep. 28, 2016 (KR) 10-2016-0124457

(51) Int. Cl. C12N 5/0783; C12N 13/00; A61K 35/17; A61P 35/00

(52) U.S. Cl. CPC C12N 5/0646; C12N 13/00; A61K 2035/124; A61P 35/00; A61K 35/17

(57) ABSTRACT

Provided is a method for preparing natural killer cell with high efficiency using irradiated peripheral blood mononuclear cells, more particularly to a method for proliferating highly activated NK cells using a combination of irradiated peripheral blood mononuclear cells (PBMCs) and a CD16 antibody and an anti-cancer cell therapeutic composition containing the natural killer cells (NK cells) prepared thereby. Further provided is a method for large-scale proliferation of activated NK cells with high efficiency using a combination of irradiated peripheral blood mononuclear cells (PBMCs) and a CD16 antibody without the use of cancer cells or genetically modified feeder cells having safety issues as feeder cells. The highly purified and highly cytotoxic NK cells proliferated in large quantities can be used as an active ingredient of a cancer immunotherapeutic composition.

### TECHNICAL FIELD

The present invention relates to a method for preparing natural killer cells (NK cells) using irradiated peripheral blood mononuclear cells (PBMCs), more particularly to a method for preparing NK cells using irradiated PBMCs and anti-CD16 antibody, and an anti-cancer cell therapeutic composition comprising the NK cells.

### BACKGROUND ART

Natural killer (NK) cells constitute approximately 10-15% of the lymphocytes in humans and are usually defined as CD3⁻CD56⁺ cells [1]. The primary function of NK cells is immune surveillance of the body. Unlike T cells, NK cells play an important role in early immune responses by removing viral infections and cancer without recognizing specific antigens [2-4]. In particular, NK cells can effectively inhibit the growth of cancer stem-like cells as well as tumor growth and metastasis in the human body. The effector function of NK cells is determined by the balance between activating and inhibitory receptor signals which are induced by binding with their ligands expressed from cancer cells [5]. An NK cell activating signal is mediated by various NK cell receptors, including CD16 (Fcγ-receptor), natural killer group 2D (NKG2D), 2B4, and natural cytotoxicity receptors (NCRs; NKp30, NKp44, NKp46, and NKp80) [5, 6]. Therefore, NK cells directly remove the target cells by binding with activation ligands expressed from the tumor cells and secreting cytotoxic granules such as perforin and granzymes, etc. In contrast, an NK cell inhibitory signal mainly is mediated by killer cell immunoglobulin-like receptors (KIRs) and CD94/NKG2A, which recognize major histocompatibility complex (MHC) class I molecules on target cells. Thus, MHC class I-deficient cancer or transformed cells are highly sensitive to NK cells [5, 7].

NK cell activation is synergistically augmented by coengagement of other activating receptors such as NKG2D and 2B4 [8, 9]. NKG2D is a key member of activating receptors present on the surface of NK cells and performs an important function in the elimination of target cells [10, 11]. There are various kinds (MICA, MICB, ULBP1, ULBP2, and ULBP3) of NKG2D ligands and they show various expression patterns in different target cells. Among them, the MHC class I-related chain A and B (MICA/B) and UL-16-binding proteins (ULBPs) are induced by various stressors, including heat shock, ionizing radiation, oxidative stress, and viral infection [12, 13].

2B4 (CD244) is one of the well-known NK cell-activating receptors. The ligand of 2B4, CD48, is broadly expressed on hematopoietic cells, including NK cells themselves. 2B4-CD48 interactions predominantly induce NK cell activation through recruiting the small adaptor SAP bound to the tyrosine kinase Fyn [8, 9]. Recently, it was reported that 2B4-mediated signaling is intimately involved in augmenting NK cell activation and proliferation both in vitro and in vivo [14].

NK cells express CD16 (FcγRIII), a low-affinity receptor for IgG; this receptor is responsible for antibody-dependent cellular cytotoxicity (ADCC). ADCC is one of the major factors for the efficacy of antibody-based cancer therapies [15]. Most CD56^{dim} NK cells show high-density expression of CD16 but CD56^{bright} NK cells lack the expression of CD16 or show low-density expression [1]. In particular, CD16 has a unique ability to induce NK cell activation without additional receptor signals [9].

Recently, it was reported that individual receptor-ligand interactions are not sufficient to induce efficient activation of resting NK cells. Thus, combinations of NK cell-activating receptors are needed to induce NK cell activation and eliminate the target cell (infected cells, cancer cells, etc.) efficiently [8, 9, 16].

Lately, it is believed that one of the key factors in the success of NK cell-based cancer immunotherapy is dependent on obtaining a sufficient number of highly cytotoxic NK cells [17, 18]. NK cells can be generated from cord blood, bone marrow, embryonic stem cells, and peripheral blood. In the early studies, a variety of cytokines (IL-15, IL-21, IL-12, and IL-18) have been used to expand NK cells, but these cytokines were not very effective. Recently, for NK cell activation and expansion, cancer cell lines, genetically modified K562 cells (artificial antigen-presenting cells with membrane-bound MICA, 4-1BBL, membrane-bound IL-15 and IL-21), or Epstein-Barr virus-transformed lymphoblastoid cell lines have been used as feeder cells after being irradiated [19-23]. Even though these methods have made large-scale NK cell expansion possible, they have brought up safety issues because they used cancer cell-based feeder cells.

In the present invention, we used irradiated autologous peripheral blood mononuclear cells (PBMCs) (IrAPs) instead of cancer cell-based feeder cells for large-scale expansion of cytotoxic NK cells with high safety and cytotoxicity. Radiation upregulates NKG2D ligands and CD48 (a 2B4 ligand) in human PBMCs. Nonetheless, irradiated autologous PBMCs alone did not induce efficient expansion of NK cell. To overcome these problems, we used an anti-CD16 monoclonal antibody (mAb) for potent activation of resting NK cells and added irradiated PBMCs for providing a suitable environment (activating receptor-ligand interactions and soluble growth factors) for the NK cell expansion. These expanded NK cells showed potent cytotoxicity against various cancer cells in vitro and efficiently controlled cancer progression in animal models of human colon and lung cancer. Thus, the proposed method provides safe and robust expansion of highly purified cytotoxic human NK cells for adoptive immunotherapy without using cancer cell-based feeder cells.

### SUMMARY OF INVENTION

The object of the present invention is to provide a method for expanding cytotoxic human NK cells for adoptive immunotherapy efficiently and safely without using cancer cell-based feeder cells.

Another object of the present invention is to provide an anti-cancer cell therapeutic composition comprising the NK cells expanded according to the method of the present invention.

Other objects and advantage of the present invention will become apparent from the detailed description to follow taken in conjugation with the appended claims and drawings.

### DETAILED DESCRIPTION

In an aspect, the present invention provides a method for preparing highly purified activated natural killer cells (NK cells) using feeder cells, wherein irradiated peripheral blood mononuclear cells (PBMCs) are used as the feeder cells and the NK cells are treated with a CD16 antibody.

In the present invention, ‘feeder cells’ mean the cells that provide nutrients to natural killer cells (NK cells) and help the activation and proliferation of NK cells via intercellular contact, growth factors, etc. And, ‘activated natural killer cells’ mean the NK cells having the immune activity capable of attacking abnormal cells such as cancer cells. In addition, ‘highly purified’ means that the purity (or proportion) of natural killer cells (NK cells) is very high, specifically the proportion of the NK cells being 98% or higher and the proportion of contaminant cells such as T cells being lower than 2%, more specifically the proportion of the NK cells being 99% or higher and the proportion of contaminant cells such as T cells being lower than 1%. In the present invention, irradiated peripheral blood mononuclear cells (PBMCs) are abbreviated as IrAP and the CD16 antibody is abbreviated as αCD16.

Specifically, the present invention provides a method for preparing highly purified activated natural killer cells (NK cells), which includes the following steps:

a) a step of isolating peripheral blood mononuclear cells (PBMCs) from human peripheral blood;

b) a step of isolating natural killer cells (NK cells) from the isolated peripheral blood mononuclear cells;

c) a step of preparing feeder cells by irradiating the peripheral blood mononuclear cells (PBMCs) remaining after isolating the natural killer cells; and

d) a step of culturing the isolated natural killer cells (NK cells) and the prepared feeder cells in a CD16 antibody-immobilized incubator.

Specifically, the present invention provides a method for preparing highly purified activated natural killer cells (NK cells) wherein, in the step b), the natural killer cells (NK cells) are isolated from the isolated peripheral blood mononuclear cells using a magnetic microbead-attached antibody and a column. As the antibody, a CD56 (NK cell) antibody may be used for positive selection and CD3 (T cells), CD14 (monocyte) and CD19 (B cell) antibodies may be used for negative selection.

Specifically, the present invention provides a method for preparing highly purified activated natural killer cells (NK cells) wherein, in the step c), the feeder cells are prepared by mixing the peripheral blood mononuclear cells (PBMCs) remaining after isolating the NK cells well in physiological saline or a medium and irradiating at 23-27 Gy. According to the examples of the present invention (see Result 1 and FIG. 1A), T cells were clearly detectable during NK cell activation and proliferation for radiation doses of 5, 10, 15 and 20 Gy, whereas T cells were effectively inactivated at a radiation dose of 25 Gy. Specifically, when the radiation dose was 25 Gy, NK cells were observed with high purity (99% or higher) and T cells were hardly observed (lower than 1%). If T cells remain during the NK cell activation and proliferation, there is a high risk of graft-versus-host disease (GVHD) when the NK cells are used as a cell therapeutic agent. Formerly, it was thought that irradiation of about 20 Gy would be enough for preventing the proliferation of feeder cells and a higher radiation dose would hinder the role as feeder cells due to necrocytosis. However, the inventors of the present invention have found out that irradiation of 23-27 Gy allows for the performance of the role as feeder cells during the NK cell activation and proliferation while completely eliminating contamination by T cells.

Specifically, the present invention provides a method for preparing highly purified activated natural killer cells (NK cells) wherein, in the step d), the isolated NK cells are treated with NKG2D and 2B4 antibodies. According to the examples of the present invention (see Result 2 and FIG. 2), it was confirmed that the proliferation of NK cells treated with IrAP and αCD16 was significantly inhibited by treatment with a NKG2D- or 2B4-blocking antibody. In particular, the NK cell proliferation was remarkably inhibited by treatment with the NKG2D- and 2B4-blocking antibodies together. Accordingly, it can be seen that the proliferation of NK cells is strongly induced by synergistic combinations of the activating receptors CD16, NKG2D and 2B4.

The present invention provides a method for preparing highly purified activated natural killer cells (NK cells) wherein the irradiated peripheral blood mononuclear cells (PBMCs) inhibits the activation of T cells and increases the expression of NKG2D ligands and CD48. According to the examples of the present invention (see Result 1 and FIGS. 1A-1C), it was confirmed that irradiation inhibits the T cell activity of peripheral blood mononuclear cells (PBMCs) and increases the expression of NKG2D ligands and CD48.

The present invention provides a method for preparing highly purified activated natural killer cells (NK cells) wherein the proliferation of the NK cells is promoted by a combination of the irradiated peripheral blood mononuclear cells (PBMCs) and the CD16 antibody. According to the examples of the present invention (see Result 2 and FIG. 2A), although IrAP induced the proliferation of NK cells, the proliferation of NK cells was remarkably enhanced by a combination of IrAP with αCD16.

The present invention provides a method for preparing highly purified activated natural killer cells (NK cells) wherein the proliferation of the NK cells is strongly induced by a synergistic combination of activating receptors CD16, NKG2D and 2B4. According to the examples of the present invention (see Result 2 and FIG. 2), it was confirmed that, when a group in which NK cells are cultured by treating with a combination of IrAP and αCD16 is treated with a NKG2D- or 2B4-blocking antibody, the proliferation of NK cells is inhibited significantly. In particular, it was confirmed that the proliferation of NK cells is inhibited remarkably when they are treated with NKG2D- and 2B4-blocking antibodies at the same time. Accordingly, it can be seen that a synergistic combination of activating receptors CD16, NKG2D and 2B4 strongly induce the proliferation of NK cells.

The present invention provides a method for preparing highly purified activated natural killer cells (NK cells) wherein the expression of activating receptors of the NK cells is increased by a combination of the irradiated peripheral blood mononuclear cells (PBMCs) and the CD16 antibody. According to the examples of the present invention (see Result 3 and FIG. 3), the NK cells proliferated by a combination of irradiated peripheral blood mononuclear cells (IrAP) and an anti-CD16 monoclonal antibody (αCD16) showed significantly increased NKG2D, DNAM-1, 2B4, NKp30, NKp44 and NKp46 receptors as compared to resting NK cells. In addition, the expression of CD56, CD16, DNAM-1, 2B4, NKp30, NKp44 and NKp46 was significantly increased as compared to the NK cells proliferated by IrAP or αCD16 alone.

The present invention provides a method for preparing highly purified activated natural killer cells (NK cells) wherein CD107a is highly expressed in the NK cells proliferated by a combination of the irradiated peripheral blood mononuclear cells (PBMCs) and the CD16 antibody. According to the examples of the present invention (see Result 4 and FIG. 4), the expression of CD107a in the NK cells proliferated by a combination of IrAP and αCD16 was increased by 6.1 times or more as compared to resting NK cells.

The present invention provides a method for preparing highly purified activated natural killer cells (NK cells) wherein the NK cells proliferated by a combination of the irradiated peripheral blood mononuclear cells (PBMCs) and the CD16 antibody strongly increases the secretion of IFN-γ upon stimulation by target cancer cells. According to the examples of the present invention (see Result 5 and FIG. 5), the NK cells proliferated by a combination of IrAP and αCD16 showed increased secretion of IFN-γ than the NK cells proliferated by IrAP or αCD16 alone.

The present invention provides a method for preparing highly purified activated natural killer cells (NK cells) wherein the NK cells proliferated by a combination of the irradiated peripheral blood mononuclear cells (PBMCs) and the CD16 antibody show strongly increased antitumor cytotoxicity against target cancer cells. According to the examples of the present invention (see Result 6 and FIG. 6), it was confirmed that the NK cells proliferated by a combination of IrAP and αCD16 show higher antitumor cytotoxicity against target cancer cells than the NK cells proliferated by IrAP or αCD16 alone.

The present invention provides a method for preparing highly purified activated natural killer cells (NK cells) wherein the NK cells proliferated by a combination of the irradiated peripheral blood mononuclear cells (PBMCs) and the CD16 antibody show strong antitumor effect in a cancer-induced mouse model. According to the examples of the present invention (see Result 7 and FIG. 7), it was confirmed that the NK cells proliferated by a combination of IrAP and αCD16 strongly induce antitumor effect in colon and lung cancer NOD/SCID mouse models. In particular, it was confirmed that a combination with irradiation can further improve the antitumor effect of the proliferated NK cells by increasing the expression of NKG2D ligands in cancer cells.

In another aspect, the present invention provides an anti-cancer cell therapeutic composition containing highly purified activated natural killer cells (NK cells) prepared by the method according to the present invention as an active ingredient.

The present invention provides an anti-cancer cell therapeutic composition, wherein the cancer may be any cancer known to be treated by activated natural killer cells (NK cells). For example, the cancer may be colon cancer or lung cancer.

The cell therapeutic composition of the present invention may contain a pharmaceutically acceptable carrier commonly used in formulation, such as lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenozate, propyl hydroxybenozate, talc, magnesium stearate, mineral oil, etc., although not being limited thereto. In addition to these ingredients, the cell therapeutic composition of the present invention may further contain a lubricant, a wetting agent, a sweetener, a flavor, an emulsifier, a suspending agent, a preservative, etc. Suitable pharmaceutically acceptable carriers and formulations are described in detail in Remington's Pharmaceutical Sciences (19th ed., 1995).

The cell therapeutic composition of the present invention may be prepared into a unit dosage form using a pharmaceutically acceptable excipient according to a method that can be easily employed by those of ordinary skill in the art to which the present invention belongs. It may be prepared into a formulation in the form of a suspension in a cell freezing solution or a suspension in a buffer solution and may further contain a stabilizer.

The cell therapeutic composition of the present invention may be administered parenterally by intravenous injection, subcutaneous injection, intraabdominal injection, transdermal administration, etc.

An appropriate administration dosage of the cell therapeutic composition of the present invention may be determined variously considering such factors as formulation method, administration type, the age, body weight and sex of a patient, administration time and administration route. Specifically, the administration dosage may be 1×10⁹ to 10×10⁹ cells per administration.

In the examples of the present invention, NK cells were proliferated using a combination of irradiated peripheral blood mononuclear cells (PBMCs) and a CD16 antibody and their anticancer effects were tested. The experimental result is analyzed as follows.

Analysis of Results

NK cells play an important role in innate immune response and are considered a promising therapeutic option for various malignant diseases [18, 25, 26]. Because NK cells constitute only a small portion of peripheral blood lymphocytes, a sufficient number of the cells should be obtained for clinical application. Although various methods have been developed for large-scale proliferation of NK cells in vitro [19-23], it is important to control their growth and to ensure that no viable cells are mixed with the proliferated NK cells because most methods involve cancer cells or genetically modified cells as feeder cells. Therefore, the number and putridity of the proliferated NK cells should be considered as important factors in the large-scale proliferation of NK cells for clinical application.

In the present invention, a new method for large-scale proliferation of NK cells was developed using an αCD16 monoclonal antibody and IrAP as feeder cells. Feeder cells provide a suitable environment for the proliferation of NK cells through various mechanisms, including cell-cell interactions and production of growth factors [27, 28]. CD16 (FcγRIII) is associated with the ITAM (immunoreceptor tyrosine-based activation motif)-containing FcεRI γ chain and CD3ξ chain [29]. Unlike other NK cell receptors, CD16 has the unique ability to activate resting NK cells without an additional activation signal. And, activation of NK cells by CD16 can be further enhanced by other receptor signals [9]. Human NKG2D is associated with DAP10, which contains a tyrosine-based signaling motif (YINM) [30, 31]. Several studies have suggested that NKG2D stimulation induces strong activation of NK cells [32-35]. NKG2D is one of very important activating receptors and provides a coactivation signal to pre-existing other activation signals, such as CD16, NKp46 and 2B4 [9, 36]. The results of the present invention also suggest that NKG2D is one of the key activation factors of NK cells in terms of antitumor cytotoxicity against target cancer cells.

A recent study reported that irradiated (20 Gy) and αCD3 monoclonal antibody- and rhIL-2-stimulated peripheral blood mononuclear cells (PBMCs) as feeder cells express significantly larger amounts of ULBP1-3 as compared to fresh PBMCs, while MIC-A/B expression is not significantly altered [37]. In the present invention, a radiation dose of 25 Gy was used to inactivate lymphocytes in PBMCs. The irradiated PBMCs showed significantly increased MIC-A/B expression, in addition to ULBP1-3 expression, as compared to a control group (unirradiated PBMCs). In addition, increased expression of CD48, the ligand of the 2B4 activating receptor, was observed in the irradiated PBMC. Importantly, when irradiated PBMCs as feeder cells were cultured together with NK cells, the proliferation of T cells was not observed during the culturing. If the T cells proliferate, the purity of NK cells is decreased and immune rejection may occur during allotransplantation. It was reported that the radiation dose of 25 Gy is suitable to effectively inactivate lymphocytes [38]. In the present invention, it was demonstrated that the radiation dose of 25 Gy increases the expression of various NKG2D ligands and CD48 while inhibiting the proliferation of T cells contained in PBMCs.

2B4 (CD244) is expressed mostly in NK cells and is bound to CD48 which is expressed in various hematopoietic cells including T and NK cells. This 2B4-CD48 binding plays a very important role in the proliferation of NK cells [14, 39]. Although irradiated PBMCs express NKG2D ligands and CD48 capable of activating resting NK cells (NK cells isolated from peripheral blood), additional activation signals are required for sufficient activation. Unlike T cells, NK cells do not have a dominant activating receptor except for the ADCC induced by CD16. Thus, NK cell activation is regulated by combinations of synergistic receptors. Particularly, co-crosslinking of CD16 with NKG2D is reported to further enhance the Ca²⁺ flux, cytokine production and cytotoxicity toward cancer cells [9, 40]. Thus, it is thought that combinations of different activating receptor signals may strongly induce NK cell activation, including cytotoxicity against cancer cells, cytokine production, cell proliferation, etc. In the present invention, it was demonstrated using blocking antibodies specific to each receptor that the proliferation of NK cells is induced by the synergistic combinations of activating receptors CD16, NKG2D and 2B4. It was confirmed that, when NK cells cultured using IrAP and αCD16 are treated with a NKG2D- or 2B4-blocking antibody, the proliferation of the NK cells is inhibited significantly. In particular, it was confirmed that the proliferation of NK cells is inhibited remarkably when they are treated with NKG2D- and 2B4-blocking antibodies at the same time.

In the present invention, irradiated autologous PBMCs (IrAP) alone were insufficient to effectively induce the proliferation of resting NK cells. Therefore, in the present invention, a new method using a combination of the irradiated autologous PBMCs (IrAP) and the αCD16 monoclonal antibody for proliferating highly purified cytotoxic NK cells in large quantities in vitro was developed. Although NK cells are activated by IL-2, the NK cells could not be proliferated in large quantities in vitro with IL-2 alone. Although the NK cells activated by the αCD16 monoclonal antibody or IrAP show significantly increased proliferation as compared to IL-2 alone, this method was insufficient for large-scale proliferation of NK cells required for clinical application (low proliferation requires more blood drawing from the patient). In contrast, a combination of the αCD16 monoclonal antibody and IrAP remarkably increased the proliferation of NK cells (5,000 times or higher). Most importantly, the proliferation of NK cells was higher than the sum of the proliferation of NK cells by the αCD16 monoclonal antibody and IrAP separately. This result points to a synergistic effect of the αCD16 monoclonal antibody and IrAP in the proliferation of NK cells. On the 21st day of culturing, the NK cells proliferated by a combination of the αCD16 monoclonal antibody and IrAP showed a purity of 98% or higher and the proliferation of T cells was hardly detected (less than 1%). In addition, the proliferated NK cells showed significantly increased expression of activating receptors such as NKG2D, NKp30, NKp44, NKp46, 2B4, DNAM-1, etc. and also showed increased IFN-γ secretion and CD107a expression when stimulated with target cancer cells. These results would have affected the higher cytotoxicity against target cancer cells as compared to other culture conditions. In the present invention, the in vivo activity of the NK cells proliferated by the αCD16 monoclonal antibody and IrAP was investigated using colon and lung cancer NOD/SCID mouse models. The administered NK cells significantly inhibited tumor growth in both colon and lung cancer and this effect was further enhanced by the combination with irradiation. This result may be associated with the NKG2D ligands increased by the irradiation. Irradiation can increase the expression of various immunologically important molecules that alter the immunogenicity of cancer cells [42, 44]. Recently, it has been reported that irradiation can upregulate the NKG2D ligand, which enhances the sensitivity of various cancer cells to NK cell-mediated cytotoxicity [43, 44]. To conclude, it was demonstrated using the colon and lung cancer NOD/SCID mouse models that the NK cells proliferated by the αCD16 monoclonal antibody and IrAP show strong antitumor activity in vivo, too, and this effect is further enhanced by the combination with irradiation.

In the present invention, the cytotoxic activity of the proliferated NK cells was increased when the target cancer cells lacked the expression of MHC class I or had higher expression of NKG2D ligands. Nevertheless, the cytotoxicity was not completely inhibited by blocking of the receptor NKG2D. DNAM-1, 284, NKp30, NKp44, NKp46 or other unknown NK cell receptors may have affected the cytotoxic activity in this case [9, 40, 45, 46]. The NK cells proliferated by a combination of the αCD16 monoclonal antibody and IrAP strongly expressed the receptors inducing activation and this led to increased death of target cancer cells.

NK cells have two main effector functions: a direct cytotoxic effect and activation of other cells by secreting cytokines, etc. CD107a is known as a marker of degranulation of cytotoxic T cells or NK cell after stimulation [24, 47]. A previous study reported that CD107a expression correlates closely with NK cell functional activity such as cytokine secretion and cell lysis [24]. Activated NK cells can secrete various cytokines such as IFN-γ, TNF-α, etc. In particular, IFN-γ performs critical functions in antiviral defense, immunoregulation, antitumor responses, or the like [48, 49]. Thus, the functional activity of the NK cells proliferated by a combination of the αCD16 monoclonal antibody and IrAP was demonstrated by means of the degranulation marker CD107a, IFN-γ secretion and antitumor cytotoxicity against target cancer cells.

Overall, cell-cell communication is crucial for the coordination of cellular activation and proliferation. NK cells use various combinations of synergistic receptors for the activation and proliferation. The combination of receptor signals such as CD16, NKG2D and 2B4 has a very important impact on the downstream pathways for strongly activating and proliferating NK cells. Therefore, the inventors of the present invention developed a new method for culturing NK cells in large quantities in vitro using a combination of IrAP and the αCD16 monoclonal antibody under GMP conditions without the use of risky cancer cells or genetically modified feeder cells as feeder cells. This method allows for proliferation of highly purified and highly cytotoxic NK cells for cancer immunotherapy in large quantities.

### Advantageous Effects

The present invention provides a method for large-scale proliferation of activated NK cells with high efficiency using a combination of irradiated peripheral blood mononuclear cells (PBMCs) and a CD16 antibody without the use of cancer cells or genetically modified feeder cells having safety issues as feeder cells. The highly purified and highly cytotoxic NK cells proliferated in large quantities can be used as an active ingredient of a cancer immunotherapeutic composition.

### BRIEF DESCRIPTION OF THE DRAWINGS

The above and other objects, features and other advantages of the present invention will be more clearly understood from the following detailed description taken in conjunction with the accompanying drawings, in which;

FIG. 1A shows a result of irradiating PBMCs with various radiation doses (5, 10, 15, 20 and 25 Gy), culturing them with NK cells and measuring the proportion of NK cells and T cells by flow cytometry.

FIG. 1B shows a result of irradiating PBMCs at a radiation dose of 25 Gy, measuring the expression of NKG2D ligands with time by flow cytometry and representing the relative expression ratio as compared to before the radiation.

FIG. 1C shows a result of irradiating PBMCs at a radiation dose of 25 Gy, measuring the expression of CD48 with time by flow cytometry and representing the relative expression ratio as compared to before the radiation.

FIG. 2A shows a result of measuring cell proliferation with the Cell Counting Kit-8 (CCK-8) using blocking antibodies specific to each receptor in order to confirm whether the proliferation of NK cells is due to the synergistic combinations of activating receptors CD16, NKG2D and 2B4.

FIG. 2B shows a result of investigating the proliferation of NK cells for 21 days using irradiated PBMCs and an anti-CD16 monoclonal antibody (αCD16) either alone or in combination.

FIG. 3 shows a result of proliferating NK cells using irradiated PBMCs and an anti-CD16 monoclonal antibody (αCD16) either alone or in combination and then measuring the expression level of activating receptors by flow cytometry.

FIG. 4A shows a result of proliferating NK cells using irradiated PBMCs and an anti-CD16 monoclonal antibody (αCD16) either alone or in combination, culturing them with cancer cells (K562) and then measuring the expression level of CD107a by flow cytometry.

FIG. 4B shows a result of proliferating NK cells using irradiated PBMCs and an anti-CD16 monoclonal antibody (αCD116) either alone or in combination, culturing them with cancer cells (K562) and then measuring the expression level of CD1007a by flow cytometry.

FIG. 5 shows a result of proliferating NK cells using irradiated PBMCs and an anti-CD16 monoclonal antibody (αCD16) either alone or in combination, culturing them with cancer cells (K562) and then measuring IFN-γ secretion by the enzyme-linked immunospot (ELISpot) assay.

FIG. 6A shows a result of proliferating NK cells using irradiated PBMCs and an anti-CD16 monoclonal antibody (αCD16) either alone or in combination and then measuring the cytotoxicity against cancer cells (K562) by flow cytometry.

FIG. 6B shows a result of measuring the expression of NKG2D ligands in various cancer cells by flow cytometry.

FIG. 6C shows a result of proliferating NK cells using a combination of irradiated PBMCs and an anti-CD16 monoclonal antibody (αCD16) and then measuring the cytotoxicity against various cancer cells by flow cytometry.

FIG. 7A shows the antitumor effect of NK cells proliferated using a combination of irradiated PBMCs and an anti-CD16 monoclonal antibody (αCD16) in colon and lung cancer NOD/SCID mouse models.

FIG. 7B shows the expression level of NKG2D ligands in irradiated colon and lung cancer cells and the cytotoxicity of proliferated NK cells (using a combination of irradiated PBMCs and an anti-CD16 monoclonal antibody (αCD16)).

### MODE FOR INVENTION

Practical and presently preferred embodiments of the present invention are illustrated as shown in the following examples. However, it will be appreciated that those skilled in the art, on consideration of this disclosure, may make modifications and improvements within the spirit and scope of the present invention.

### Example 1. Culturing of Cancer Cell Lines

K562 (CCL-243), SW480 (CCL-288), A549 (CCL-185) and MCF-7 (HTB-22) cells were cultured in RPMI 1640 (K562, SW480, A549) or DMEM (MCF-7) supplemented with 100 U/mL penicillin, 100 μg/mL streptomycin and 10% fetal bovine serum (FBS) in a 5% CO₂ incubator maintained at 37° C.

### Example 2. Isolation and Culturing of NK Cells

1) Separation of Blood

10-50 mL of human peripheral blood was centrifuged (2000 rpm, 5 minutes). From the separated blood, the supernatant plasma and the red blood cells which settled down were discarded and the white blood cells in the middle layer were recovered. The recovered white blood cells were mixed well by adding physiological saline (normal saline) and loaded the density gradient solution Histopaque-1077. Then, peripheral blood mononuclear cells (PBMCs) were obtained by centrifuging at 400×g for 30 minutes at room temperature.

2) Isolation of NK Cells

Highly purified natural killer cells were obtained by incubating the isolated peripheral blood mononuclear cells with magnetic microbead-attached antibodies such as a CD56 antibody (for positive selection) or CD3, CD14 and CD19 antibodies (for negative selection) in a column.

3) Preparation of Feeder Cells

After the isolation of the NK cells, the remaining peripheral blood mononuclear cells (PBMCs) were mixed well in physiological saline (or a medium) and irradiated at a radiation dose of 25 Gy.

4) Preparation of Antibody-Immobilized Incubator

An anti-CD16 antibody prepared with a concentration of 1 μg/mL or higher in physiological saline was added to an incubator and the solution was allowed to spread uniformly on the bottom. 4-24 hours later, the antibody solution was removed and the incubator was washed 3 times with physiological saline to obtain an antibody-immobilized incubator.

5) Culturing of NK Cells

The natural killer cells (NK cells) and feeder cell (NK cells: feeder cells=1:1-100) isolated from the peripheral blood mononuclear cells were mixed well in a medium and added to the antibody-immobilized incubator. After adding 5-10% human serum and 500-1000 U/mL interleukin-2 (Proleukin, Chiron), the cells were cultured for 3-7 days at 37° C. in the presence of 5% CO₂. Then, the cells were transferred to an incubator with no antibody immobilized and a medium supplemented with 5-10% human serum and 500-1000 U/mL interleukin-2 (hereinafter referred to as a ‘nutrient medium’) was added. The cells were cultured for 21 days while adding the nutrient medium every 2-3 days depending on the degree of proliferation of the natural killer cells. On days 7, 14 and 21, the cells were recovered from the incubator in order to investigate the proliferation of the natural killer cells and identify surface antigens.

### Example 3. Analysis of Surface Antigens

Surface antigens on the cells were analyzed using monoclonal antibodies for flow cytometry. Fluorescence-labeled monoclonal antibodies such as anti-CD3-PE, CD48-FITC, CD56-PE-Cy5, CD16-PE, CD314 (NKG2D)-PE, HLA-ABC-FITC, CD337 (NKp30)-PE, CD336 (NKp44)-PE, CD335 (NKp46)-PE, CD226 (DNAM-1)-FITC, CD244 (2B4)-FITC, MICA-PE, MICB-PE, ULBP-1-PE, ULBP-2-PE, ULBP-3-PE, etc. were used and analysis was conducted with respect to the isotype control.

### Example 4. Confirmation of NK Cell Proliferation by Activating Receptors of NK Cells

The isolated NK cells were incubated with m1gG, NKG2D, CD244 (2B4) and NKG2D+CD244 (2B4) antibodies for 30 minutes in an incubator maintained at 37° C. and 5% CO₂ and then washed 3 times with physiological saline. The antibody-bound NK cells were seeded onto a 96-well plate or a CD16 antibody-immobilized 96-well plate to a concentration of 1×10⁵ cells/mL. Then, the NK cells were cultured after adding feeder cells. After culturing for 5 days and adding 10 μL of the CCK-8 (Cell Counting Kit-8) reagent to each well, the cells were incubated for 4 hours in an incubator maintained at 37° C. and 5% CO₂. 4 hours later, absorbance was measured at 450 nm using an ELISA reader.

### Example 5. Confirmation of NK Cell Function

1) Analysis of CD107a

NK cells were cocultured with K562 (human chronic myelogenous leukemia cell line) cells at a ratio of 1:1 in a medium supplemented with anti-CD107a-PE, BD GolgiStop™ and BD GolgiPlug™ for 4-6 hours at 37° C. in the presence of 5% CO₂. Then, the cells were centrifugally washed 3 times with physiological saline and then incubated with anti-CD56-PC5 for 20-30 minutes. Then, the expression level of CD1007a was measured by flow cytometry.

2) Analysis of Interferon Gamma (IFN-γ) by Enzyme-Linked Immunospot (ELISpot) Assay

NK cells and target cancer cells (1:10) were added to an ELISpot plate coated with a capture antibody and containing 200 μL of a nutrient medium and then incubated for 4 hours in an incubator maintained at 37° C. and 5% CO₂. After washing with physiological saline, a detection antibody was added at 100 μL per well and the plate was incubated for 2 hours at room temperature. After washing with physiological saline, a color developing reagent was added to each well and the plate was incubated in the dark. After the incubation, the color developing reaction was completed using distilled water and the plate was dried well. Finally, interferon gamma (IFN-γ) was quantified using the ELISpot reader system.

3) NK Cell-Mediated Cytotoxicity Assay

In the present invention, K562, A549, SW480 and MCF-7 cells were used as the target cancer cells of NK cells. After adding 5 μM 5-carboxyfluorescein diacetate succinmidyl ester (CFSE), the target cancer cells were incubated at 37° C. for 10 minutes in the presence of 5% CO₂. Then, the cells were centrifugally washed 2-3 times using a medium supplemented with 10% human serum. NK cells (effector cells) were cocultured with the CFSE-labeled target cancer cells at ratios of 10:1, 5:1, 2.5:1 and 1:1 in a reactor tube or a 96-well plate for 4-6 hours at 37° C. in the presence of 5% CO₂. After the culturing was completed, the tube was immediately put in ice water and 50 μg/mL propidium iodide (PI) was added. The cytotoxicity of the natural killer cells (NK cells) was analyzed by flow cytometry within 1 hour.

### Example 6. Animal Experiment of NK Cells

5-to-6-week-old nonobese diabetic/severe combined immunodeficiency (NOD/SCID) NOD.CB17-Prkdcscid/ARC mice were used for animal experiment of NK cells. SW480 human colon cancer cells (2-5×10⁶ cells) and A549 human lung cancer cells (2-5×10⁶ cells) were subcutaneously inoculated into the right thighs of the mice. When the tumor grew to a size of 50-100 mm³, irradiation was applied at 8 Gy to the right thigh using a linear accelerator (Infinity. Elekta). After the irradiation, NK cells (1-2×10⁷ cells) were injected into the tail veins of the mice. The tumor size (volume=depth×width²×0.5) was measured twice a week and the irradiation and the NK cell injection were performed 3 times at 1-week intervals. 5-FU (100 mg/kg, SW480 positive control group) and docetaxel (10 mg/kg, A549 positive control group) were administered 3 days before every NK cell injection.

Experimental Results

Result 1. Irradiation Inhibits T Cell Activity and Increases Expression of NKG2D Ligands and CD48 in Peripheral Blood Mononuclear Cells (PBMC)

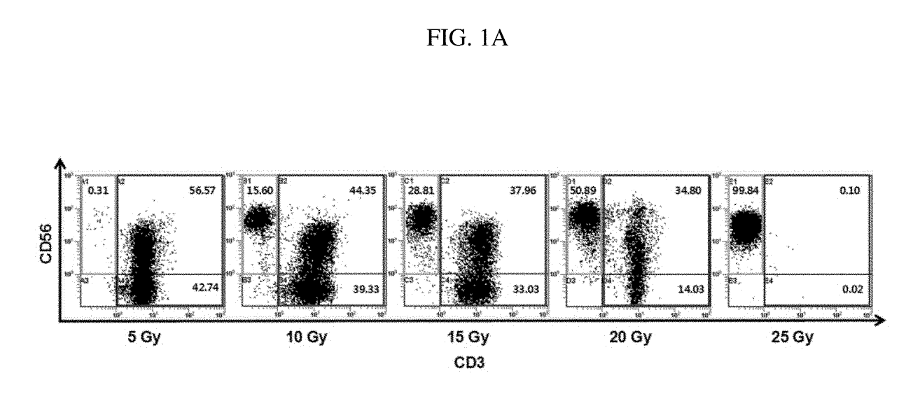

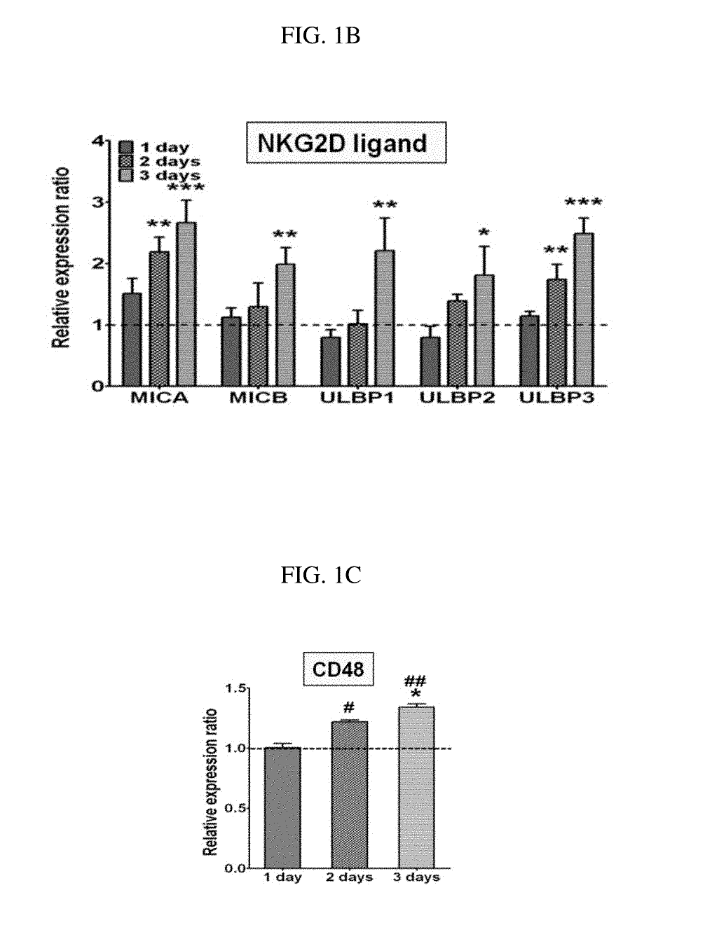

To determine the optimal dose of radiation for T-cell inactivation. PBMCs were exposed to various radiation doses (5, 10, 15, 20, 25 Gy). Then, the irradiated PBMCs were cocultured with resting NK cells (NK cells isolated from peripheral blood) for 21 days. The proportion of T cells was assessed by flow cytometry (FIG. 1A). T cells were clearly detectable during NK cell activation and proliferation after radiation doses of 5, 10, 15 and 20 Gy. However, the radiation dose of 25 Gy induced effective inactivation of T cells (T cells were hardly observed). Specifically, when NK cells were cocultured feeder cells irradiated at a dose of 25 Gy, the proportion of NK cells (green) was higher than 99% (99.84%) and the proportion of T cells (red) was less than 1% (0.12%). Therefore, 25 Gy was decided as the radiation dose for effectively inactivating T cells. To test whether irradiation induces the expression of NKG2D ligands and CD48 (2B4 ligand) in peripheral blood mononuclear cells, peripheral blood mononuclear cells isolated from donors were harvested 0, 24, 48 or 72 hours after irradiation at 25 Gy. The expression of NKG2D ligands (FIG. 1B) and CD48 (FIG. 1C) was analyzed by flow cytometry and the result was represented by mean fluorescence intensities (MFIs). Relative expression ratios were calculated by dividing the MFI value of the irradiated peripheral blood mononuclear cells by the MFI value of the fresh peripheral blood mononuclear cells. The irradiated peripheral blood mononuclear cells expressed larger amounts of MICA, ULBP3 and CD48 compared to fresh peripheral blood mononuclear cells after 2 days, whereas MICB, ULBP1 and ULBP2 expression increased 3 days after the irradiation. In addition, although the PBMCs highly expressed CD48 (2B4 ligand), this expression was further increased 2 days after the irradiation. These results indicate that the radiation dose of 25 Gy increases the expression of NKG2D ligands and CD48 in peripheral blood mononuclear cells.

FIG. 1A shows the result of irradiating PBMCs with various doses (5, 10, 15, 20 and 25 Gy), coculturing them with NK cell for 21 days and then measuring the proportions of NK cells and T cells by flow cytometry. In FIGS. 1B and 1C, the dotted lines indicate the MFI value of the peripheral blood mononuclear cells before the irradiation. Relative expression ratios were calculated by dividing the MFI value of the irradiated peripheral blood mononuclear cells by the MFI value of the fresh peripheral blood mononuclear cells. Statistical significance: *P<0.05, **P<0.005, ***P<0.0005.

Result 2. A Synergistic Combination of Activating Receptors CD16, NKG2D and 2B4 Strongly Induces Proliferation of NK Cells

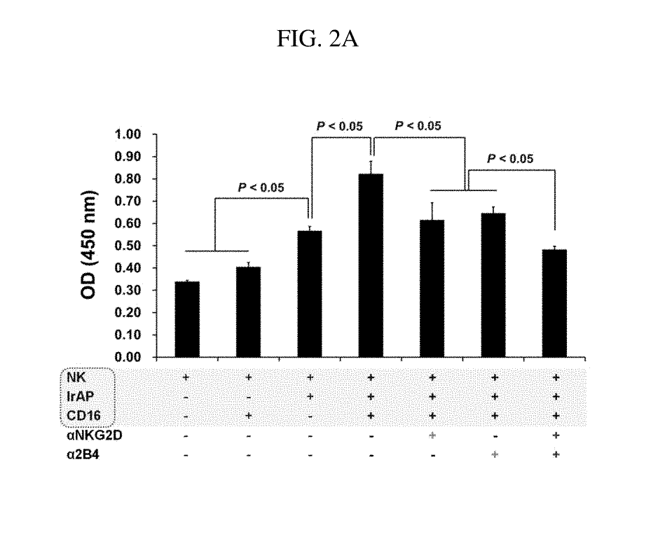

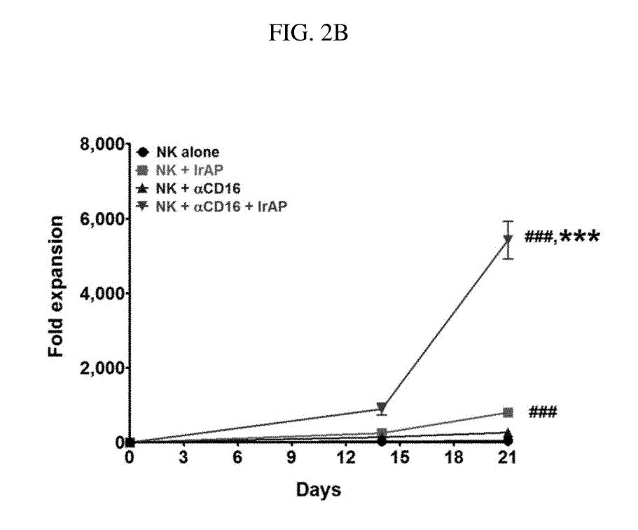

To examine the effect of a combination of irradiated peripheral blood mononuclear cells (IrAP; cells in which NKG2D and 2134 are expressed) and an anti-CD16 monoclonal antibody (αCD16) on the proliferation of NK cells, resting NK cells (NK cells isolated from peripheral blood) from five donors were isolated and irradiated. αCD16 was coated onto a plate to a concentration of 1 μg/mL or higher in advance and resting NK cells and IrAPs were cultured under Good Manufacturing Practices (GMP) conditions. First, it was investigated whether the NK cell proliferation was due to the synergistic combinations of activating receptors CD16, NKG2D and 2B4 by the Cell Counting Kit-8 (CCK-8) assay using blocking antibodies specific for each receptor. Although IrAP strongly induced the proliferation of NK cells, the proliferation of NK cells was further enhanced by a combination of IrAP and αCD16. However, the proliferation of NK cells was relatively low when αCD16 was used alone as compared to IrAP or IrAP+αCD16 (FIG. 2A). It was confirmed that, for the NK cells treated with a combination of IrAP and αCD16, treatment with NKG2D- or 2B4-blocking antibody resulted in significantly decreased proliferation of NK cells. In particular, the proliferation of NK cells was more strongly inhibited by the treatment with the NKG2D- and 2B4-blocking antibodies at the same time. These results indicate that the proliferation of NK cells is induced by coactivation of receptors NKG2D and 2B4, and this effect was more strongly induced by synergistic combinations of the receptors CD16, NKG2D and 2B4. In particular, it was confirmed that this effect is more strongly induced by the synergistic combinations of the activating receptors CD16, NKG2D and 2B4. As shown in FIG. 2B, IL-2 alone failed to significantly induce the proliferation of NK cells (42.8±3.8 fold), whereas the NK cells stimulated with IrAP or αCD16 were significantly proliferated as compared to IL-2 alone (IrAP; 794±115.6 fold, αCD16; 259.2±44.4 fold). In particular, the NK cells stimulated with a combination of IrAP and αCD16 were remarkably proliferated (5421.6±505.4 fold). This interaction points to a synergistic effect of IrAP and αCD16 in the proliferation of NK cells. Thus, it was demonstrated that a combination of IrAP and αCD16 synergistically enhances the proliferation of NK cells. In FIG. 2, statistical significance: ###P<0.0005 (#; NK alone versus other groups). ***P<0.0005 (*; NK+IrAP versus NK+αCD16 or NK+αCD16+IrAP).

Result 3. A Combination of Irradiated Peripheral Blood Mononuclear Cells (IrAP) with an anti-CD16 Monoclonal Antibody (αCD16) Increases the Expression of NK Cell-Activating Receptors

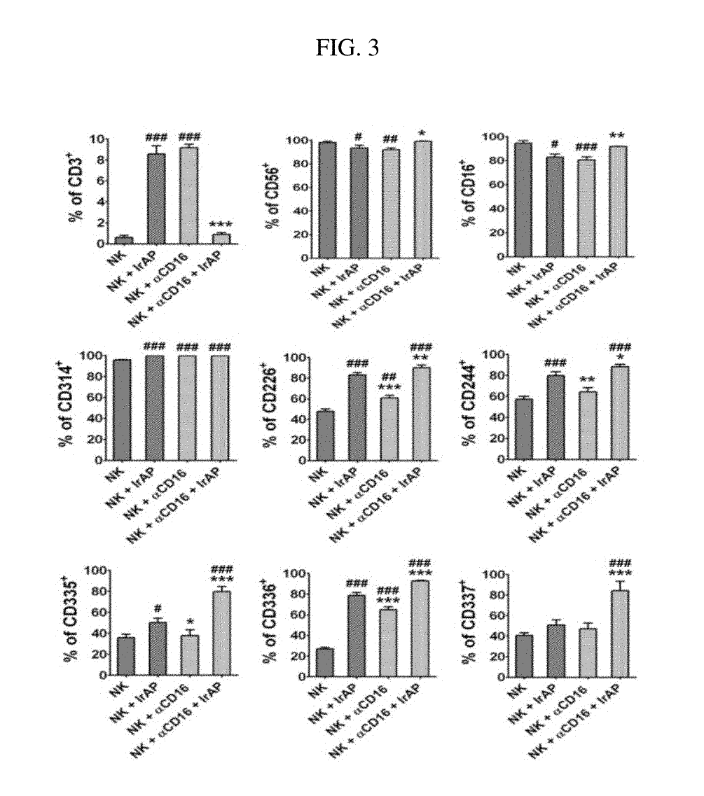

The phenotypic differences between NK cells isolated from peripheral blood (resting NK cell) and proliferated NK cells were evaluated. These cells were analyzed by flow cytometry and then the expression levels of CD3, CD56, CD16, NKG2D (CD314), NKp30 (CD337), NKp44 (CD336), NKp46 (CD335), 2184 (CD244) and DNAM-1 (CD226) were compared. As shown in FIG. 3, the NK cells proliferated by a combination of IrAP and αCD16 showed significantly increased expression of activating receptors (NKG2D, DNAM-1, 2B4, NKp30, NKp44 and NKp46) as compared to the resting NK cells. Nonetheless, CD3, CD56 and CD16 expression levels were not significantly changed. In addition, this proliferation method produced significant differences in CD3, CD56, CD116, DNAM-1, 2B4, NKp30, NKp44 and NKp46 as compared to the NK cells expanded by either IrAP or αCD16 alone. The NK cells proliferated by either IrAP or αCD16 showed significant differences in NKG2D, DNAM-L, 2B4/NKp46 (NK cells proliferated by αCD16) and NKp44 as compared to the resting NK cells. In contrast, CD56 and CD16 expression levels significantly decreased and NKp30 showed no significant change. Furthermore, there were significant differences in the expression levels of DNAM-1, 284, NKp44 and NKp46 between the NK cells proliferated by IrAP and αCD16. In addition, the NK cells proliferated by a combination of IrAP and αCD16 had negligible T-cell (CD3) contamination as compared to the NK cells proliferated by either IrAP or αCD16 alone. T cells were hardly detectable during the proliferation (<1%). Thus, these results indicate that the combination of IrAP and αCD16 may further increase the expression of the NK cell-activating receptors in the proliferation of NK cells. In FIG. 3, statistical significance: #P<0.05, ##P<0.005, ###P<0.0005 (#; NK alone versus other groups). *P<0.05, **P<0.005, ***P<0.0005 (*; NK+IrAP versus NK+αCD16 or NK+αCD16+IrAP.

Result 4. CD107a is Highly Expressed in NK Cells Proliferated by a Combination of IrAP and αCD16

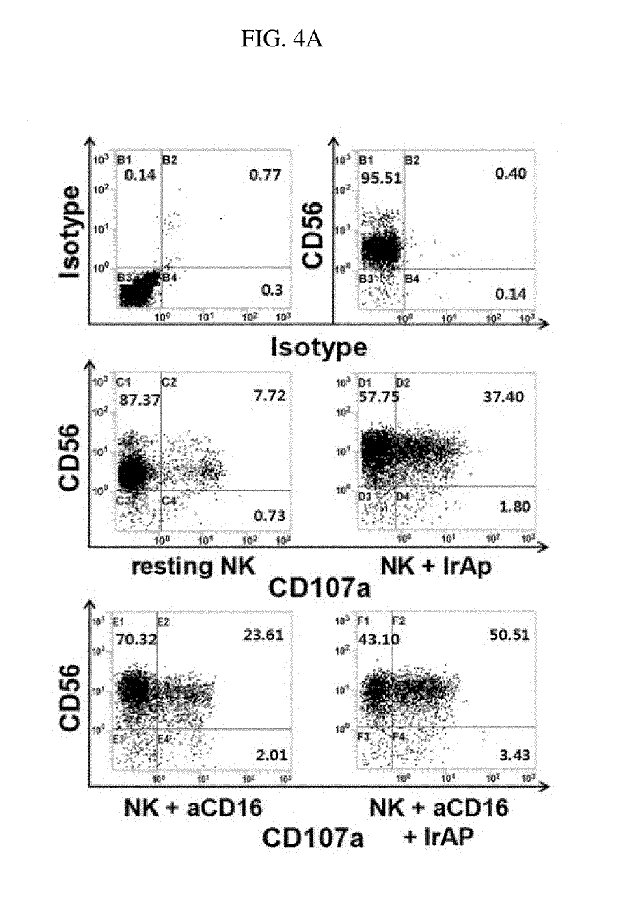

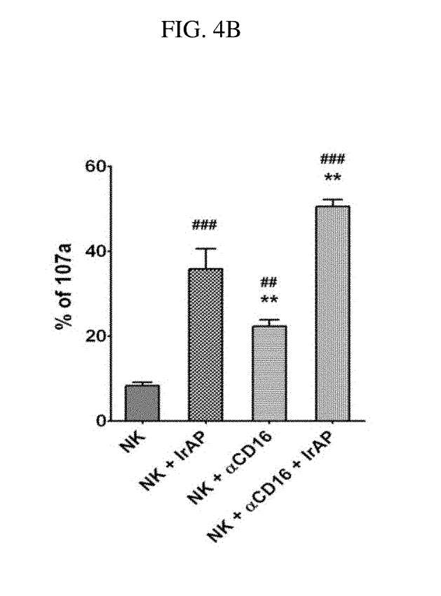

It is known that CD107a expression correlates closely with the activity of NK cells [24]. It was determined whether the degranulation marker CD107a was expressed on the surface of the NK cells proliferated under various conditions. The proliferated NK cells were incubated with K562 cells as target cancer cells. After 4 hours of incubation in the presence of monensin and an anti-CD107a monoclonal antibody, NK cells were stained by adding anti-CD3 and anti-CD56 monoclonal antibodies. As shown in FIG. 4, the resting NK cells expressed very little CD107a on the cell surface upon contact with the K562 cells, but CD107a expression on the surface of the NK cells (proliferated under various culture conditions) increased more than 2.7-fold as compared to the resting NK cells. In particular, the CD107a expression on the surface of NK cells proliferated by a combination of IrAP and αCD16 was 6.1-fold as compared to the resting NK cells. Thus, these results indicate that the NK cells proliferated by a combination of IrAP and αCD16 may further increase the expression of CD107a caused by stimulation with target cancer cells. In FIG. 4, statistical significance: ##P<0.005, ###P<0.0005 (#; NK alone versus other groups). **P<0.005, (*; NK+IrAP versus NK+αCD16 or NK+αCD16+IrAP).

Result 5. NK Cells Proliferated by a Combination of IrAP and αCD16 Strongly Increase IFN-γ Secretion after Stimulation with Target Cancer Cells

The IFN-γ secretion of NK cells after stimulation with target cancer cells was evaluated. The IFN-γ ELISpot assay was performed on resting NK cells (NK cells isolated from peripheral blood) and proliferated NK cells using K562 cells as target cancer cells. The resting NK cells secreted relatively low amounts of IFN-γ after stimulation with K562 cells, but the NK cells proliferated under various culture conditions strongly increased IFN-γ secretion. Specifically, the NK cells proliferated by a combination of IrAP and αCD16 secreted larger amounts of IFN-γ than did the NK cells proliferated by either IrAP or αCD16. These results may be related to the CD107a expression. Thus, these findings indicate that the NK cells proliferated by a combination of IrAP and αCD16 may further increase IFN-γ secretion after stimulation with target cancer cells.

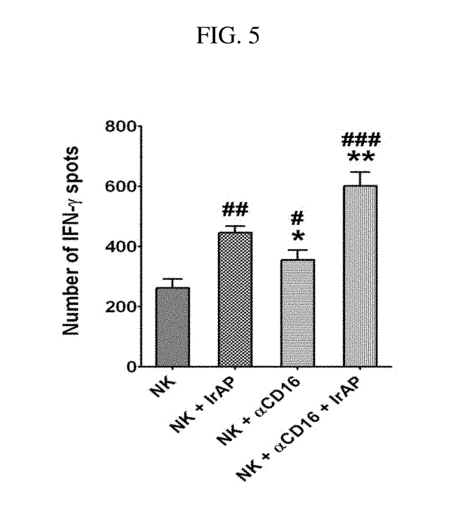

In FIG. 5, statistical significance: ##P<0.005, ###P<0.0005 (#; NK alone versus other groups). *P<0.05, **P<0.005, (*; NK+IrAP versus NK+αCD16 or NK+αCD16+IrAP).

Result 6. NK Cells Proliferated by a Combination of IrAP and αCD16 Show Strongly Enhanced Antitumor Cytotoxicity Against Target Cancer Cells

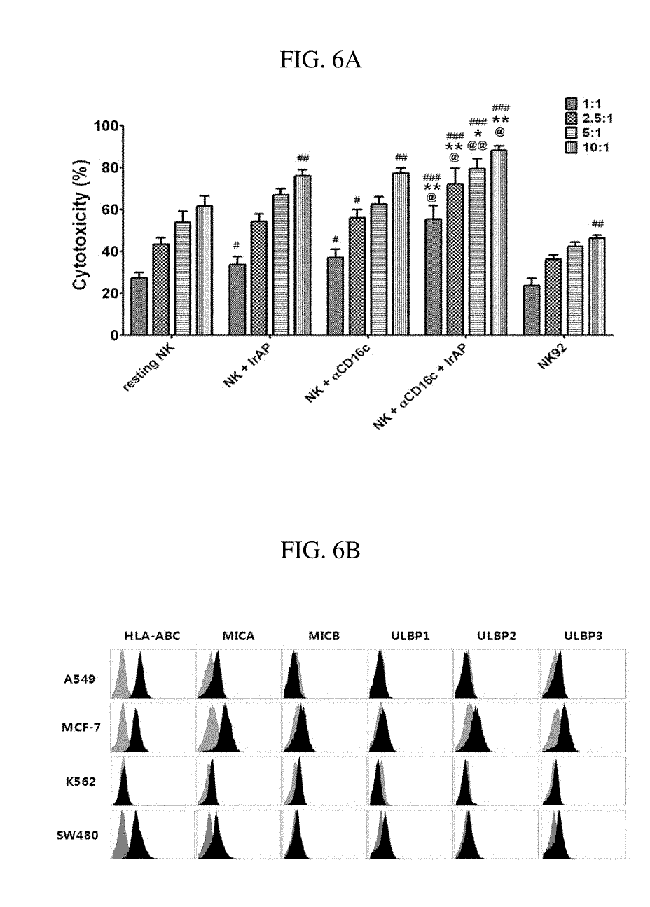

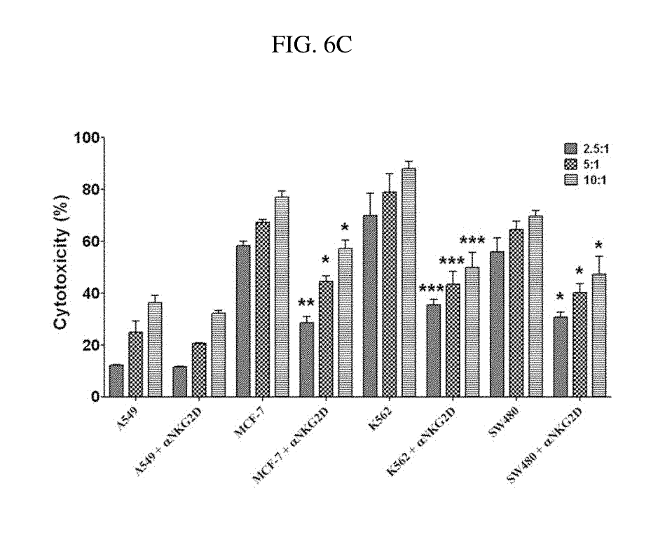

The antitumor cytotoxicity of NK cells proliferated using an MHC class I-negative cell line (K562) and MHC class I-positive cell lines (MCF-7, A549, and SW480) was evaluated. As shown in FIG. 6A, the antitumor cytotoxicity against target cancer cells was significantly elevated in the proliferated NK cells compared to resting NK cells (NK cells isolated from peripheral blood) and NK-92 cells. In particular, the NK cells proliferated by a combination of IrAP and αCD16 showed higher antitumor cytotoxicity than did the NK cells proliferated by either the IrAP or αCD16. These results may be related to CD107a expression and IFN-γ secretion. As shown in FIG. 6B, the most NK-sensitive target cancer cells, K562 cells, expressed NKG2D ligands but did not express MHC class I. The A549 cells weakly expressed NKG2D ligands but MHC class I was strongly expressed. They were weakly sensitive to the NK cells proliferated by a combination of IrAP and αCD16. Although the MCF-7 and SW480 cells expressed MHC class I, these cells strongly expressed NKG2D ligands as compared to other cancer cells. They were moderately sensitive to the NK cells proliferated by a combination of IrAP and αCD16. The NK-sensitive target cancer cells (K562, MCF-7 and SW480) tended to highly express NKG2D ligands or weakly express MHC class I as compared to the NK-resistant target cells (A549). To evaluate the effect of NKG2D ligands on the antitumor cytotoxicity of NK cells, the NK cells proliferated by a combination of IrAP and αCD16 were cocultured with target cancer cells in the presence of a NKG2D-blocking antibody (used to inhibit the biding between the NKG2D receptors of NK cells and the ligands of the target cancer cells). Blocking of the receptor NKG2D resulted in a substantial reduction in antitumor cytotoxicity against all target cancer cells except for the A549 cells, which show low expression of NKG2D ligands (FIG. 6C). These results indicate that the NK cells proliferated by a combination of IrAP and αCD16 exert increased antitumor cytotoxicity against target cancer cells and NKG2D is one of the important factors in the activation of NK cells. In FIG. 6, statistical significance: #P<0.05, ##P<0.005, ###P<0.0005 (#; NK alone versus other groups). *P<0.05, **P<0.005, ***P<0.0005 (*; NK+IrAP versus NK+αCD16 or NK+αCD16+IrAP, target tumor cell versus NKG2D blocking). @P<0.05, @@P<0.005 (@; NK+αCD16 versus NK+IrAP or NK+αCD16+IrAP).

Result 7. NK Cell Proliferated by a Combination of IrAP and αCD16 Strongly Induce Antitumor Effect in Colon and Lung Cancer NOD/SCID Mouse Models

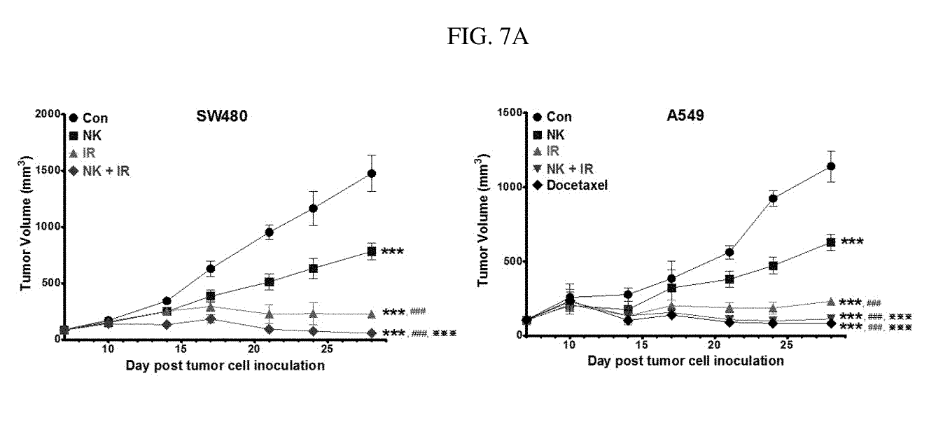

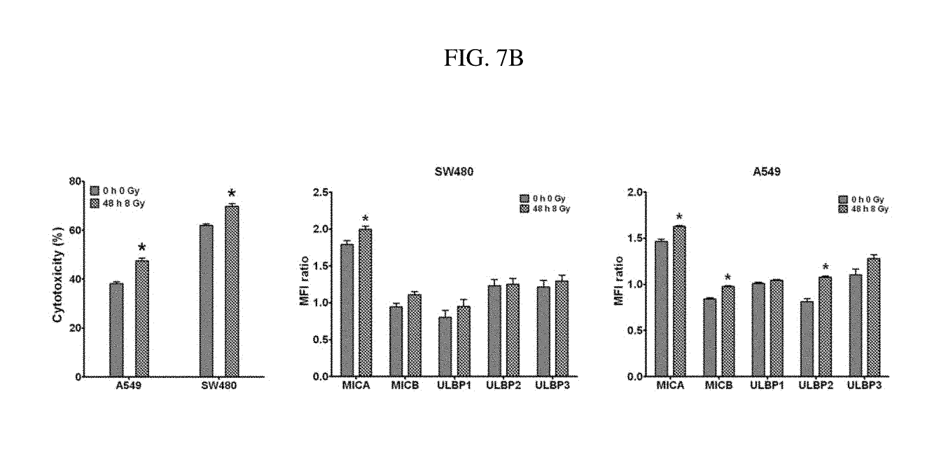

The antitumor effect of NK cells proliferated by a combination of IrAP and αCD16 was evaluated using colon and lung cancer NOD/SCID mouse models. SW480 (human colon cancer) cells and A549 (human lung cancer) cells were subcutaneously inoculated into the right thighs of NOD-SCID mice. Irradiation was applied at a radiation dose of 8 Gy to the tumor in the right thigh of the mice. Then, the proliferated NK cells proliferated by a combination of IrAP and αCD16 were injected into the tail vein. 5-FU and docetaxel were injected 3 days before every NK injection. The NK cells proliferated by a combination of IrAP and αCD16 significantly inhibited tumor growth in both colon cancer (SW480) and lung cancer (A549) NOD/SCID mouse models. In particular, the antitumor effect of the NK cells was further enhanced by the combined treatment with irradiation (FIG. 7A). The irradiation increased the expression of NKG2D ligands in the SW480 and A549 cells and further enhanced the cytotoxicity of the NK cell against target cancer cells (FIG. 7B). These results demonstrate the in vivo antitumor effect of the NK cells proliferated by a combination of IrAP and αCD16 in colon cancer (SW480) and lung cancer (A549) NOD/SCID mouse models. In particular, the combined treatment with irradiation could further enhance the in vivo antitumor activity of the NK cells by increasing the expression of NKG2D ligands in cancer cells. In FIG. 7, statistical significance: *P<0.05, **P<0.005, ***P<0.0005 (*; con versus other groups, 0 h 0 Gy versus 48 h 8 Gy). ###P<0.0005 (#; NK versus other groups). ※※※P<0.0005 (※; IR versus NK+IR or docetaxel).

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

### REFERENCES

1. Cooper M A, Fehniger T A, Caligiuri M A. The biology of human natural killer-cell subsets. Trends Immunol. November 2001; 22(11): 633-640.
2. Trinchieri G. Biology of natural killer cells. Adv Immunol. 1989; 47: 187-376.
3. Robertson M J, Ritz J. Biology and clinical relevance of human natural killer cells. Blood. Dec. 15, 1990; 76(12): 2421-2438.
4. Caligiuri M A. Human natural killer cells. Blood Aug. 1, 2008; 112(3): 461-469.
5. Lanier L L. NK cell recognition. Annu Rev Immunol. 2005; 23: 225-274.
6. Moretta A, Bottino C, Vitale M, et al. Activating receptors and coreceptors involved in human natural killer cell-mediated cytolysis. Annu Rev Immunol. 2001; 19: 197-223.
7. Long E O. Regulation of immune responses through inhibitory receptors. Annu Rev Immunol. 1999; 17: 875-904.
8. Bryceson Y T, March M E, Ljunggren H G, Long E O. Activation, coactivation, and costimulation of resting human natural killer cells. Immunol Rev. December 2006; 214: 73-91.
9. Bryceson Y T, March M E, Ljunggren H G, Long E O. Synergy among receptors on resting NK cells for the activation of natural cytotoxicity and cytokine secretion. Blood. Jan. 1, 2006; 107(1): 159-166.
10. Raulet D H. Roles of the NKG2D immunoreceptor and its ligands. Nat Rev Immunol. October 2003; 3(10): 781-790.
11. Bottino C, Castriconi R, Moretta L, Moretta A. Cellular ligands of activating NK receptors. Trends Immunol April 2005; 26(4): 221-226.
12. Vivier E, Tomasello E, Paul P. Lymphocyte activation via NKG2D: towards a new paradigm in immune recognition? Curr Opin Immunol. June 2002; 14(3): 306-311.
13. Watzl C. The NKG2D receptor and its ligands-recognition beyond the “missing self”? Microbes Inect. January 2003; 5(1): 31-37.
14. Kim T J, Kim M, Kim H M, Lim S A, Kim E O, Kim K, Song K H, Kim J, Kumar V, Yee C, et al.: Homotypic NK cell-to-cell communication controls cytokine responsiveness of innate immune NK cells. Sci Rep 2014, 4: 7157.
15. Sulica A, Morel P, Metes D, Herbennan R B. Ig-binding receptors on human NK cells as effector and regulatory surface molecules. Int Rev Immunol. June 2001; 20(3-4): 371-414.
16. Andre P, Castriconi R, Espeli M, et al. Comparative analysis of human NK cell activation induced by NKG2D and natural cytotoxicity receptors. Eur J Immunol. April 2004; 34(4): 961-971.
17. Koepsell S A, Miller J S, McKenna D H, Jr. Natural killer cells: a review of manufacturing and clinical utility. Transfusion. February 2013; 53(2): 404-410.
18. Cheng M, Chen Y. Xiao W. Sun R, Tian Z. NK cell-based immunotherapy for malignant diseases. Cell Mol Immunol. May 2013; 10(3): 230-252.
19. Lim S A, Kim T J, Lee J E, et al. Ex vivo expansion of highly cytotoxic human NK cells by cocultivation with irradiated tumor cells for adoptive immunotherapy. Cancer Res. Apr. 15, 2013; 73(8): 2598-2607.
20. Gong W, Xiao W. Hu M, et al. Ex vivo expansion of natural killer cells with high cytotoxicity by K562 cells modified to co-express major histocompatibility complex class I chain-related protein A, 4-1 BB ligand, and interleukin-15. Tissue Antigens. December 2010; 76(6): 467-475.
21. Fujisaki H, Kakuda H, Shimasaki N, et al. Expansion of highly cytotoxic human natural killer cells for cancer cell therapy. Cancer Res. May 1, 2009; 69(9): 4010-4017.
22. Denman C J, Senyukov V V, Somanchi S S, et al. Membrane-bound IL-21 promotes sustained ex vivo proliferation of human natural killer cells. PLoS One. 2012; 7(1): e30264.
23. Berg M, Lundqvist A, McCoy P. Jr., et al. Clinical-grade ex vivo-expanded human natural killer cells up-regulate activating receptors and death receptor ligands and have enhanced cytolytic activity against tumor cells. Cytotherapy. 2009; 11(3): 341-355.
24. Alter G, Malenfant J M, Altfeld M. CD107a as a functional marker for the identification of natural killer cell activity. J Immunol Methods. November 2004; 294(1-2): 15-22.
25. Barao I, Murphy W J. The immunobiology of natural killer cells and bone marrow allograft rejection. Biol Blood Marrow Transplant. December 2003; 9(12): 727-741.
26. Passweg J R, Koehl U, Uharek L, Meyer-Monard S, Tichelli A. Natural-killer-cell-based treatment in haematopoietic stem-cell transplantation. Best Pract Res Clin Haematol. 2006; 19(4): 811-824.
27. Ehmann U K, Stevenson M A, Calderwood S K, DeVries J T. Physical connections between feeder cells and recipient normal mammary epithelial cells. Exp Cell Res. Aug. 25, 1998; 243(1): 76-86.
28. Miller J S, Oelkers S, Verfaillie C, McGlave P. Role of monocytes in the expansion of human activated natural killer cells. Blood. Nov. 1, 1992; 80(9): 2221-2229.
29. Bottino C, Moretta L, Pende D, Vitale M, Moretta A. Learning how to discriminate between friends and enemies, a lesson from Natural Killer cells. Mol Immunol. July 2004; 41(6-7): 569-575.
30. Wu J, Song Y, Bakker A B, et al. An activating immunoreceptor complex formed by NKG2D and DAP10. Science. Jul. 30, 1999; 285(5428): 730-732.
31. Rosen D B, Araki M, Hamerman J A, Chen T, Yamamura T, Lanier L L. A Structural basis for the association of DAP12 with mouse, but not human, NKG2D. J Immunol. Aug. 15, 2004; 173(4): 2470-2478.
32. Gross O, Grupp C, Steinberg C, et al. Multiple ITAM-coupled NK-cell receptors engage the Bc110/Malt1 complex via Carma1 for NF-kappaB and MAPK activation to selectively control cytokine production. Blood. Sep. 15, 2008; 112(6): 2421-2428.
33. Sutherland C L, Chalupny N J, Schooley K, VandenBos T, Kubin M, Cosman D. UL16-binding proteins, novel MHC class 1-related proteins, bind to NKG2D and activate multiple signaling pathways in primary NK cells. J Immunol. Jan. 15, 2002; 168(2): 671-679.
34. Jamieson A M, Diefenbach A, McMahon C W, Xiong N, Carlyle J R, Raulet D H. The role of the NKG2D immunoreceptor in immune cell activation and natural killing. Immunity. July 2002; 17(1): 19-29.
35. Billadeau D D, Upshaw J L, Schoon R A, Dick C J, Leibson P J. NKG2D-DAP10 triggers human NK cell-mediated killing via a Syk-independent regulatory pathway. Nat Immunol. June 2003; 4(6): 557-564.
36. Guerra N. Tan Y X, Joncker N T, et al. NKG2D-deficient mice are defective in tumor surveillance in models of spontaneous malignancy. Immunity. April 2008; 28(4): 571-580.
37. Ahn Y O, Kim S, Kim T M, Song E Y, Park M H, Heo D S. Irradiated and activated autologous PBMCs induce expansion of highly cytotoxic human NK cells in vitro. J Immunother. September 2013; 36(7): 373-381.
38. Pelszynski M M, Moroff G, Luban N L, Taylor B J, Quinones R R. Effect of gamma irradiation of red blood cell units on T-cell inactivation as assessed by limiting dilution analysis: implications for preventing transfusion-associated graft-versus-host disease. Blood. Mar. 15, 1994; 83(6): 1683-1689.
39. Assarsson E, Kambayashi T, Schatzle J D, et a. NK cells stimulate proliferation of T and NK cells through 2B4/CD48 interactions. J Immunol. Jul. 1, 2004; 173(1): 174-180.
40. Kim T J, Kim M, Kim H M, et al. Homotypic NK cell-to-cell communication controls cytokine responsiveness of innate immune NK cells. Sci Rep. 2014; 4: 7157.
41. Tomasello E, Blery M, Vely F, Vivier E. Signaling pathways engaged by NK cell receptors: double concerto for activating receptors, inhibitory receptors and NK cells. Semin Immunol. April 2000; 12(2): 139-147.
42. Formenti S C, Demaria S. Systemic effects of local radiotherapy. Lancet Oncol. July 2009; 10(7): 718-726.
43. Friedman E J. Immune modulation by ionizing radiation and its implications for cancer immunotherapy. Curr Pharm Des. 2002; 8(19): 1765-1780.
44. Kim J Y, Son Y O, Park S W, et al. Increase of NKG2D ligands and sensitivity to NK cell-mediated cytotoxicity of tumor cells by heat shock and ionizing radiation. Exp Mol Med. Oct. 31, 2006; 38(5): 474-484.
45. Gasser S, Orsulic S, Brown E J, Raulet D H. The DNA damage pathway regulates innate immune system ligands of the NKG2D receptor. Nature. Aug. 25, 2005; 436(7054): 1186-1190.
46. Voskens C J, Watanabe R, Rollins S, Campana D, Hasumi K, Mann D L. Ex-vivo expanded human NK cells express activating receptors that mediate cytotoxicity of allogeneic and autologous cancer cell lines by direct recognition and antibody directed cellular cytotoxicity. J Exp Clin Cancer Rev. 2010; 29:134.
47. Lim O, Lee Y, Chung H, et al. GMP-compliant, large-scale expanded allogeneic natural killer cells have potent cytolytic activity against cancer cells in vitro and in vivo. PLoS One. 2013; 8(1): e53611.
48. Schroder K, Hertzog P J, Ravasi T, Hume D A. Interferon-gamma: an overview of signals, mechanisms and functions. J Leukoc Biol. February 2004; 75(2): 163-189.
49. Street S E, Cretney E, Smyth M J. Perforin and interferon-gamma activities independently control tumor initiation, growth, and metastasis. Blood. Jan. 1, 2001; 97(1): 192-197.

1. A method for preparing highly purified activated natural killer cells (NK cells) using feeder cells, wherein irradiated peripheral blood mononuclear cells (PBMCs) are used as the feeder cells and the NK cells are treated with a CD16 antibody.

2. The method for preparing highly purified activated natural killer cells (NK cells) according to claim 1, which comprises:
a) isolating peripheral blood mononuclear cells (PBMCs) from human peripheral blood;
b) isolating natural killer cells (NK cells) from the isolated peripheral blood mononuclear cells;
c) preparing feeder cells by irradiating the peripheral blood mononuclear cells (PBMCs) remaining after isolating the natural killer cells; and
d) culturing the isolated natural killer cells (NK cells) and the prepared feeder cells in a CD16 antibody-immobilized incubator.

3. The method for preparing highly purified activated natural killer cells (NK cells) according to claim 2, wherein, in b), the natural killer cells (NK cells) are isolated from the isolated peripheral blood mononuclear cells using a magnetic microbead-attached antibody and a column.

4. The method for preparing highly purified activated natural killer cells (NK cells) according to claim 2, wherein, in c), the feeder cells are prepared by mixing the peripheral blood mononuclear cells (PBMCs) remaining after isolating the NK cells well in physiological saline or a medium and irradiating at 23-27 Gy.

5. The method for preparing highly purified activated natural killer cells (NK cells) according to claim 2, wherein, in d), the isolated NK cells are treated with NKG2D and 2B4 antibodies.

6. The method for preparing highly purified activated natural killer cells (NK cells) according to claim 1, wherein the irradiated peripheral blood mononuclear cells (PBMCs) inhibits the activation of T cells and increases the expression of NKG2D ligands and CD48.

7. The method for preparing highly purified activated natural killer cells (NK cells) according to claim 1, wherein the proliferation of the NK cells is promoted by a combination of the irradiated peripheral blood mononuclear cells (PBMCs) and the CD16 antibody.

8. The method for preparing highly purified activated natural killer cells (NK cells) according to claim 1, wherein the proliferation of the NK cells is strongly induced by a synergistic combination of activating receptors CD16, NKG2D and 2B4

9. The method for preparing highly purified activated natural killer cells (NK cells) according to claim 1, wherein the expression of activating receptors of the NK cells is increased by a combination of the irradiated peripheral blood mononuclear cells (PBMCs) and the CD16 antibody.

10. The method for preparing highly purified activated natural killer cells (NK cells) according to claim 1, wherein CD107a is highly expressed in the NK cells proliferated by a combination of the irradiated peripheral blood mononuclear cells (PBMCs) and the CD16 antibody.

11. The method for preparing highly purified activated natural killer cells (NK cells) according to claim 1, wherein the NK cells proliferated by a combination of the irradiated peripheral blood mononuclear cells (PBMCs) and the CD16 antibody strongly increases the secretion of IFN-γ upon stimulation by target cancer cells.

12. The method for preparing highly purified activated natural killer cells (NK cells) according to claim 1, wherein the NK cells proliferated by a combination of the irradiated peripheral blood mononuclear cells (PBMCs) and the CD16 antibody show strongly increased antitumor cytotoxicity against target cancer cells.

13. The method for preparing highly purified activated natural killer cells (NK cells) according to claim 1, wherein the NK cells proliferated by a combination of the irradiated peripheral blood mononuclear cells (PBMCs) and the CD16 antibody show strong antitumor effect in a cancer-induced mouse model.

14. An anti-cancer cell therapeutic composition comprising highly purified activated natural killer cells (NK cells) prepared by the method according to claim 1 as an active ingredient.
